(19) United States
(12) Patent Application Publication
(10) Pub. No.: US 2025/0345344 A1
(43) Pub. Date: Nov. 13, 2025

(54) NOVEL NRF2 ACTIVATOR AND USE THEREOF

(71) Applicant: SHANGHAI INSTITUTE FOR BIOMEDICAL AND PHARMACEUTICAL TECHNOLOGIES, Xuhui District, Shanghai (CN)

(72) Inventors: Wei ZHENG, Shanghai (CN); Juan LI, Shanghai (CN); Jianxing CHEN, Shanghai (CN); Ting ZHANG, Shanghai (CN); Ting HUANG, Shanghai (CN); Liangkang CHEN, Shanghai (CN); Haijing CAO, Shanghai (CN)

(21) Appl. No.: 18/861,307

(22) PCT Filed: Apr. 21, 2023
(86) PCT No.: PCT/CN2023/089682

§ 371 Date: Oct. 29, 2024

(30) Foreign Application Priority Data
Jul. 8, 2022 (CN) 202210805342.5

(51) Int. Cl. A61K 31/55; A61P 25/28; C07D 403/12

(52) U.S. Cl. CPC A61K 31/55; A61P 25/28; C07D 403/12

(57) ABSTRACT

Provided is use of a compound represented by Formula I or a pharmaceutically acceptable salt thereof as an Nrf2 activator. In Formula I, m is independently selected from an integer from 0 to 4, and R₁ and R₂ are independently selected from hydrogen, hydroxyl, halogen, C₁₋₃ alkyl, halogen-substituted C₁₋₃ alkyl, or C₁₋₃ alkoxy. The Nrf2 activator is used for preparing a medicament for the treatment and/or prevention of diseases mediated by the Keap1-Nrf2/ARE signaling pathway and/or diseases mediated by oxidative stress.

### TECHNICAL FIELD

The present invention belongs to the field of chemical and pharmaceutical technology, and particularly relates to a pharmaceutical use of a novel class of nuclear factor erythroid-2 (E2) related factor 2 (Nrf2) activator, which is expected to be used for preparing a medicament for the prevention or treatment of diseases mediated by the Keap1-Nrf2/ARE signaling pathway or diseases mediated by oxidative stress (OS).

### BACKGROUND

Nuclear factor erythroid-2 related factor 2 (Nrf2) is a key transcription factor involved in antioxidant defense, and it regulates approximately 250 genes involved in cellular homeostasis, including antioxidant proteins, detoxifying enzymes, drug transporters, and various cell protective proteins.

Under normal physiological conditions, a negative regulatory protein Kelch-like ECH-associated protein 1 (Keap1) locks Nrf2 in the cytoplasm, preventing it from entering the nucleus, and interacts with ubiquitin ligase 3 (Cullin 3) to mediate Nrf2 ubiquitination degradation to maintain the homeostasis of Nrf2 in the cell. Various stimuli can cause changes in the spatial conformation of Keap1 and its dimer, allowing Nrf2 to dissociate from Keap1 into the nucleus and thus exhibiting biological functions (Keum, Y. S.; Choi, B. Y. Molecular and chemical regulation of the Keap1-Nrf2 signaling pathway. Molecules, 2014, 19 (7): 10074-10089).

Antioxidant response element (ARE) is located in an upstream regulatory region of phase II detoxifying enzyme and antioxidant kinase genes, and is a specific DNA promoter binding sequence. Nrf2 is an activating factor of this sequence. When activated Nrf2 dissociates from Keap1 and enters the nucleus, it binds to Maf proteins (including Maf G, Maf K, Maf F) to form heterodimers, and then binds to ARE, causing genes regulated by ARE to start transcription (reading), thereby initiating the expression of protective genes such as phase II detoxifying enzymes and antioxidant stress proteins (Hayes, J. D.; Dinkova Kostova, A. T. The Nrf2 regulatory network provides an interface between redox and intermediate metabolism. Trends in Biochemical Sciences, 2014, 39 (4): 199-218). Nrf2 is crucial in the antioxidant and anti-inflammatory defense mechanisms of the body, and can serve as a potential therapeutic target for diseases caused by oxidative stress (OS) and inflammation.

OS is caused by increased production of reactive oxygen species (ROS) in the body, weakened antioxidant capacity, and damage to lipids, proteins, and DNA. The pathogenesis of diseases such as amyotrophic lateral sclerosis (ALS), multiple sclerosis (MS), Friedrich's ataxia, and apoplexia is closely related to OS. Regarding these diseases, a promising strategy at present is to maintain a redox balance in the body by regulating the Keap1-Nrf2/ARE signaling pathway (Brandes, M. S.; Gray, N. E. NRF2 as a therapeutic target in neurodegenerative diseases. ASN Neuro, 2020, 12:1-23). Research has shown that as age increases, the brain's OS intensifies, accompanied by a decrease in Nrf2 expression; while a large number of clinical studies have confirmed that Nrf2 activators can effectively treat these diseases, such as dimethyl fumarate (DMF), which is a known Nrf2 activator and was approved by the FDA as a first-line treatment for MS in 2013; such as Curcumin, as an Nrf2 activator, playing a role in the treatment of ALS (Cuadrado, A.; Rojo, A. I.; Wells, G, et al. Therapeutic targeting of the NRF2 and KEAP1 partnership in chronic diseases. Nat Rev Drug Discov, 2019, 18 (4): 295-317).

OS is also the pathogenesis of various reproductive system diseases, and abnormal follicular development, premature ovarian failure, and infertility are all related to excessive production of ROS (Lu, J; Wang, Z; Cao, J; Chen, Y; Dong, Y. A novel and comparative review on the role of oxidative stress in female reproduction. Reproductive Biology and Endocrinology. 2018, 16:80). The Keap1-Nrf2/ARE signaling pathway is involved in regulating the redox balance of the reproductive system, and can play a positive role as a potential target for preventing and treating reproductive system diseases (Yucong Ma, Aimin Yang, Shuancheng Zhang, Huilan Du. Research advances of nuclear factor erythroid 2 related factor 2/antioxidant response element signaling pathway in reproductive system. Chinese Journal of Reproduction and Contraception. 2021, 41 (12): 1154-1159).

Premature ovarian failure (POF) refers to a gynecological endocrine disease in which women with normal or delayed age of menarche and with normal development of secondary sexual characteristics, experience symptoms such as perimenopausal syndrome before the age of 40, with amenorrhea lasting for more than 6 months, atrophy of reproductive organs, and decreased sexual function, or even infertility. The incidence rate of premature ovarian failure is increasing day by day, which has become a disease seriously endangering women's physical and mental health. In recent years, studies have shown that whether premature ovarian failure is caused at the organizational, molecular, or genetic levels, it is accompanied by apoptosis of ovarian granulosa cells, which also indicates a direct relationship between granulosa cell apoptosis and premature ovarian failure (Massin, N.; Meduri, G.; Bachelot, A., et al. Evaluation of different markers of the ovarian reserve in patient presentation with pre-existing ovarian failure. Mol Cell Endocrinol. 2008, 282 (1-2): 95-100.; Na Ye, Xiaoying Dong, Donghua Li. The progress in apoptotic mechanism of ovarian granulosa cells involved in premature ovarian failure. Journal of Capital Medical University, 2014, 35 (3): 379-383).

Ovarian granulosa cells surround oocytes, provide nutrients and maturation factors for the growth and development of the oocytes, and protect the oocytes from oxidative stress damage through their own antioxidant system. Nrf2 protein, as a key antioxidant factor, is mainly expressed in ovarian granulosa cells and oocytes of secondary follicles and antral follicles, and is less expressed in primary follicles and primordial follicles. The expression level of Nrf2 is highest in the ovarian tissue of mice during the reproductive period, while lower in the ovarian tissue of young and sterilized mice; therefore it is speculated that Nrf2 is correlated with ovarian reserve and has a protective effect on ovarian reserve function (Jing Chen, Xiaosheng Lu, Jieqiang Lv. Expression and localization of Nrf2 protein in ovary of mice different ages. China Maternal and Child Health, 2017, 32 (22): 5722-5724).

At present, there are many studies on the improvement of ovarian reserve capacity by Nrf2 activators. Lycopene and glycogen synthase kinase-3 can promote the expression of downstream antioxidant enzymes SOD and GSH by upregulating Nrf2, thereby exerting antioxidant effects and improving ovarian dysfunction caused by aging and chemotherapy (Liu, X. T.; Lin, X.; Zhang, S. Y., et al. Lycopene ameliorates oxidative stress in the aging chicken ovarian via activation of Nrf2/HO-1 pathway. Aging, 2018, 10 (8): 2016-2036; Niringiyumukiza, J. D.; Cai, H. C.; Chen, L., et al. Protective properties of glycogen synthase kinase-3 inhibition against doxorubicin-induced oxidative damage to mouse ovarian reserve. Biomed Pharmacother, 2019, 116:108963). In addition, DMF, as an Nrf2 activator used to treat MS, has been shown to have a protective effect on mouse ovaries (Akino, N.; Wada Hairaike, O.; Isono, W., et al. Activation of Nrf2/Keap1 pathway by oral dimethylfumarate administration adversity oxidative stress and age associated inflammation may be delayed in the mouse ovary. Reprod Biol Endocrinol, 2019, 17 (1): 23). Epidermal growth factor (EGF) secreted by human placental mesenchymal stem cells can increase the number of primordial follicles, secondary follicles, and antral follicles in mice with premature ovarian failure, and its mechanism is related to the upregulation of Nrf2/HO-1 expression by EGF. These drugs and stem cell research are expected to play an important role in enhancing ovarian reserve capacity and improving pregnancy outcomes.

### SUMMARY OF THE INVENTION

The present invention provides a use of a compound represented by Formula I or a pharmaceutically acceptable salt thereof as an Nrf2 activator:

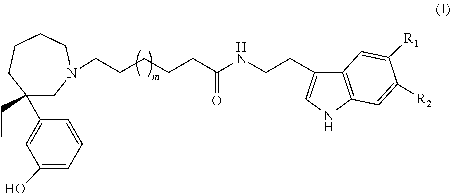

wherein
m is independently selected from an integer from 0 to 4, preferably m is 2 or 3, and R₁ and R₂ are independently selected from hydrogen, hydroxyl, halogen, C₁₋₃ alkyl, C₁₋₃ alkoxy, halogen-substituted C₁₋₃ alkyl, or halogen-substituted C₁₋₃ alkoxy; preferably, R₁ is selected from hydroxyl, halogen, or C₁₋₃ alkoxy.

In the present invention, the Nrf2 activator is used for preparing a drug for the treatment and/or prevention of diseases mediated by the Keap1-Nrf2/ARE signaling pathway and/or diseases mediated by oxidative stress. Preferably, the disease is selected from amyotrophic lateral sclerosis, Friedrich's ataxia, multiple sclerosis, apoplexia, abnormal follicular development, premature ovarian failure, and infertility.

The Nrf2 activator of the present invention has a good protective effect against neuronal damage mediated by oxidative stress (OS), can effectively inhibit neuronal apoptosis induced by hydrogen peroxide, restore mitochondrial membrane potential, inhibit caspase-3 activation, reduce ROS levels, and increase the content of glutathione and superoxide dismutase (SOD) activity. In addition, the Nrf2 activator of the present invention also has a significant protective effect on ovarian granulosa cells.

### BRIEF DESCRIPTION OF DRAWINGS

FIG. 1 shows the mechanism by which a compound Mep-S regulates the Keap1-Nrf2/ARE signaling pathway in a human SH-SY5Y neuronal injury model induced by H₂O₂, wherein a represents the levels of HO-1 protein, NQO-1 protein, Akt protein, and phosphorylated Akt protein in SH-SY5Y cells (band plot), b represents the levels of Keap1 protein, Nrf2 protein in the nucleus and cytoplasm of SH-SY5Y cells (band plot), c represents the level of HO-1 protein (semi quantitative plot), d represents the level of NQO-1 protein (semi quantitative plot), e represents the level of phosphorylated Akt protein (semi quantitative plot), and f represents the level of Keap1 protein (semi quantitative plot), g represents the level of Nrf2 protein in the nucleus (semi quantitative plot), and h represents the level of Nrf2 protein in the cytoplasm (semi quantitative plot). * P<0.05, and ** P<0.01.

FIG. 2 shows the neuroprotective effect of a compound Mep-S, wherein a represents neuronal apoptosis caused by Mep-S inhibition of H₂O₂ (flow cytometry), b represents cell apoptosis caused by Mep-S inhibition of H₂O₂ (semi quantitative plot), c represents caspase-3 activation caused by Mep-S inhibition of H₂O₂ (band plot), and d represents the level of caspase-3 activation protein (semi quantitative plot). * P<0.05, and ** P<0.01.

### DETAILED DESCRIPTION OF PREFERRED EMBODIMENTS

The present invention provides a use of a compound represented by Formula I or a pharmaceutically acceptable salt thereof as Nrf2 activator:

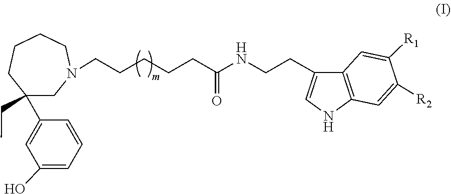

wherein
m is independently selected from an integer from 0 to 4, and
R₁ and R₂ are independently selected from hydrogen, hydroxyl, halogen, C₁₋₃ alkyl, halogen-substituted C₁₋₃ alkyl, or C₁₋₃ alkoxy.

The preparation of the compound represented by Formula I or a pharmaceutically acceptable salt thereof and the definition of each group involved in the present invention refer to CN 102816151A, which discloses the use of a series of levomeprazole derivatives (Mep-S) in the treatment of Alzheimer's disease.

The following pharmacological test examples further illustrate the present invention, but do not limit the present invention.

### Example 1

Example 1 illustrates the mechanism and neuroprotective effect of compounds Mep-S in regulating the Keap1-Nrf2/ARE signaling pathway. The protective effect and mechanism of the compounds Mep-S on nerve injury are investigated by using H₂O₂ induced human SH-SY5Y nerve cell injury model.

### Experimental Method

The culture of cells. Human neuroblastoma cells SH-SY5Y (American Type Culture Collection, Manassas, VA, USA) were cultivated in an incubator at 37° C. and a volume fraction of 5% CO₂. The culture medium contains DMEM/F-12, 2 mM glutamine, 100 U/ml penicillin, 100 μg/ml streptomycin, and 10% FBS. Cells were seeded into a 96 well plate at a density of 2×10⁴ cells per well and subjected to proliferation until 90%.

The cells were randomly divided into four groups: blank control group, hydrogen peroxide group (Veh), Mep-S group, and NAC group (a neuroprotective drug used as a positive control). The cells of Mep-S group (1 μM) and NAC group (1 mM) were pre-treated with corresponding concentrations of drugs for 1 hour. Then, except for the blank control group which was given physiological saline, the cells of all other groups were incubated with H₂O₂ (400 μM) for 24 hours.

Western blot was used to detect the expression levels of neuronal apoptosis protein (Caspase3) and Keap1-Nrf2/ARE pathway related proteins (Keap1, Nrf2, HO-1, NQO-1, etc.) in each group. The bands were visualized using enhanced chemiluminescence (Pierce, Rockford, IL, USA) and quantitative analysis was performed using Image Studio Lite 5.2.

Annexin V/PI dual staining method was used to detect the protective effect of Mep-S on cellular oxidative damage. Cell apoptosis was measured by flow cytometry using FITC Annexin V apoptosis detection kit (BD Pharmingen, Franklin Lakes, NJ, USA). The operation was performed following the instructions.

### Experimental Result

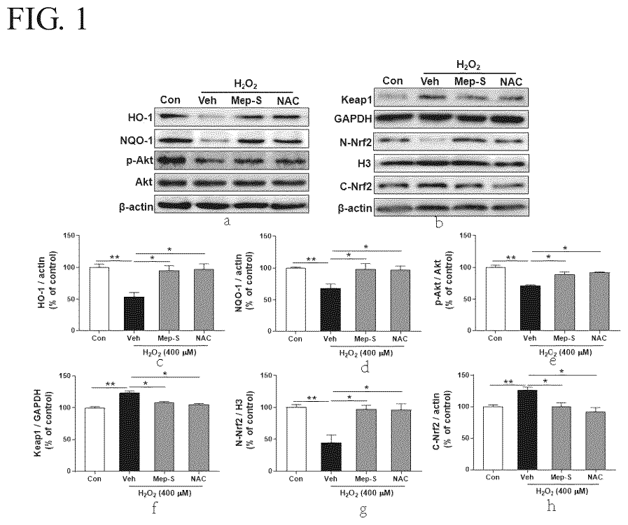

The experimental result is shown in FIG. 1. The levels of HO-1 protein and NQO-1 protein in SH-SY5Y cells damaged by H₂O₂ oxidation are significantly lower than those in the blank control group (P<0.01); meanwhile, compared with the control group, the levels of Keap 1 protein (a negative regulator of Nrf2) and Nrf2 protein in the cytoplasmic increases, while the level of Nrf2 protein in the cell nucleus decreases (P<0.01). Compared with the H₂O₂ damage group, the pretreated Mep-S group or NAC group significantly inhibits the decrease in the level of HO-1 protein, NQO-1 protein, and Nrf2 protein in the cell nucleus caused by H₂O₂ injury, as well as inhibits the increase in Keap 1 protein and Nrf2 protein in the cytoplasmic (P<0.05). Phosphatidylinositol-3-kinase (PI3K)/Akt pathway is an important upstream regulator of Nrf2 signaling that promotes Nrf2 phosphorylation and nuclear translocation (Li ST. Acta Pharmacol Sin. 2018; 39:1294-1304). We found that the phosphorylation level of Akt in SH-SY5Y cells damaged by H₂O₂ was significantly lower than that in the blank control group (P<0.01), while Mep-S or NAC intervention significantly restores its expression level (P<0.05).

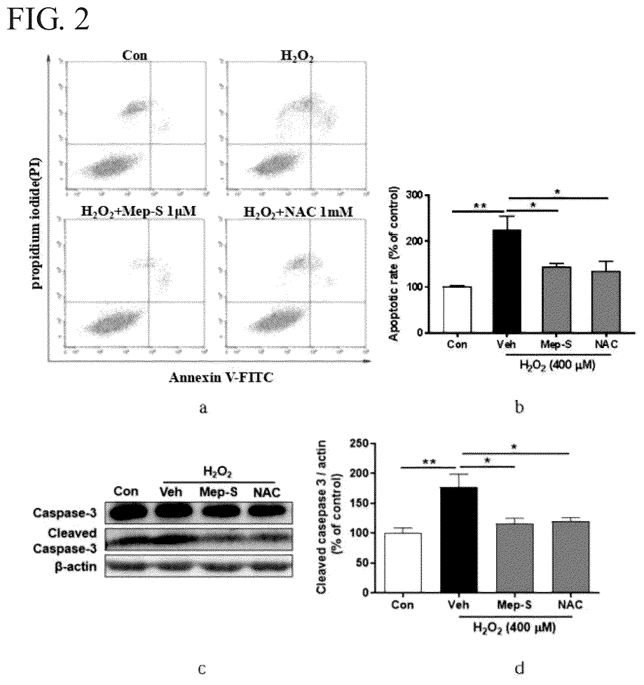

The results of Annexin V/PI dual staining method is shown in FIG. 2. The apoptosis rate of cells in the H₂O₂ injury group is 2.2 times higher than that in the control group (P<0.01). The pretreatment of Mep-S or NAC significantly reduces the apoptosis rate of SH-SY5Y cells (P <0.05). The expression of apoptotic proteins is shown in FIG. 2. It can be seen that Mep-S significantly inhibits the upregulation of activated caspase-3 levels under H₂O₂ induced injury (P<0.05).

In the above experiment, the concentration of Mep-S test substance was only one thousandth of the positive control drug NAC, but their effects are comparable, indicating that Mep-S is a potent Nrf2 activator with good protective effect against OS mediated neuronal damage and can reduce neuronal apoptosis.

### Example 2

The protective effect of compounds (C₁, i.e. Mep-S(a compound represented by Formula I, m is 3, R₁ is OH, and R₂ is H), C2 (a compound represented by Formula I, m is 3, R₁ is Cl, and R₂ is H), C3 (a compound of Formula I, m is 3, R₁ is OCH₃, and R₂ is H), C4 (a compound represented by Formula I, m is 3, R₁ is H, and R₂ is H)) on ovarian granulosa cells at a concentration of 1 μM. In this example, primary granulosa cells were collected from mice ovaries, cytotoxicity was induced using a chemotherapy drug doxorubicin (DOX) with granulosa cytotoxicity, and the protective effects of a series of test compounds on ovarian granulosa cells were tested.

### Experimental Materials

Animals: SPF grade female ICR mice, 6 mice, 21 to 23 days, body weight (14±2) g. The F1 generation of male and female mice bred in an animal room provided by Shanghai Xipu Bikai Experimental Animal Co., Ltd., with experimental animal qualification certificate number SCXK (Shanghai) 2015-0016. The feeding conditions in the animal room are: room temperature (23±2° C.), humidity 45% to 55%, 12 hours of light exposure time, and free access to water.

Reagent: CCK-8 reagent, purchased from Shanghai Rui'an Biotechnology Co., Ltd; M2 operating medium, α-MEM culture medium, and fetal bovine serum (FBS), purchased from Gibco company; Dimethyl sulfoxide (DMSO) and mineral oil, both purchased from Sigma Corporation in the United States; Doxorubicin (DOX), purchased from Shanghai Jinsui Biotechnology Co., Ltd; test compounds C1˜C4 are dissolved in methanol (10 μM), and 10 μL was taken when using.

### Experimental Method

In-vitro-Culture of granulosa cells of mice. Female ICR mice aged 21 to 23 days were euthanized by dislocation after anesthesia. After disinfecting the abdominal skin with 75% alcohol, bilateral ovaries were taken out and antral follicles were punctured under dissecting microscope to release granulosa cells into a M2 culture medium containing 20% FBS. Then the culture medium was centrifuged at 1,000 revolutions per minute for 5 minutes. Supernatant was discarded, and residue was washed three times with a culture medium, and then was placed in a 96 well plate in an alpha MEM culture medium containing 5% FBS for further culture. The alpha MEM culture medium also contains 100 U/mL penicillin, 100 U/mL streptomycin, 100 mU/mL follicle stimulating hormone, and 10 mU/mL luteinizing hormone.

After the granulosa cells in a 96 well plate prolificated to 70% to 80%, the culture medium was taken out, and 10 μL of the test drug (C1 (a compound represented by Formula I, m is 3, R₁ is OH, and R₂ is H), C2 (a compound represented by Formula I, m is 3, R₁ is Cl, and R₂ is H), C3 (a compound represented by Formula I, m is 3, R₁ is OCH₃, and R₂ is H), C4 (a compound represented by Formula I, m is 3, R₁ is H, and R₂ is H)), 10 μL of DOX with a final concentration of 25 μg/mL, and 80 μL of fresh culture medium were sequentially added. At the same time, a control well (10 μL of test drug and 90 μL of fresh culture medium) was established to observe the effect of the test drug itself on granulosa cells. A negative control well containing only granulosa cells (100 μL fresh culture medium) and a positive control well containing granulosa cells and DOX (10 μL DOX and 90 μL fresh culture medium) were also established. Four replicates were set for each test drug concentration, while 16 replicates were set for the negative and positive control groups. After 24 hours of cultivation, 10 μL of CCK-8 was added to each group and allowed to stand for 2 hours to 3 hours. The absorbance (OD) value (450 nm) of each well was measured using an enzyme-linked immunosorbent assay (ELISA) reader to calculate the survival rate of granulosa cells of mice. Statistical analysis was conducted using SPSS 16.0 statistical software. T-test was used for intragroup comparison, and multivariate analysis of variance was used for intergroup comparison, with p<0.05 indicating statistical significance.

### Experimental Result

The negative control group showed good growth of ovarian granulosa cells of mice. The positive control group showed that the addition of DOX at a final concentration of 25 μg/mL significantly inhibited the growth of granulosa cells (p<0.01), but the addition of the test drug increased the survival rate of granulosa cells in C1˜C3 groups. That indicates that the test drug has a protective effect against DOX induced toxicity in ovarian granulosa cells at a concentration of 1 μM (results shown in Table 1), with the preferred compound being C3.

TABLE 1

| Groups | Cell survival rate (%) |
|---|---|
| Positive control group (DOX) | 61.8 |
| DOX and C1 (m is 3; R₁ is OH; and R₂ is H) | 72.1 |
| DOX and C2 (m is 3; R₁ is Cl; and R₂ is H) | 69.4 |
| DOX and C3 (m is 3; R₁ is OCH₃; and R₂ is H) | 78.7 |
| DOX and C4 (m is 3; R₁ is H; and R₂ is H) | 63.9 |

1. An Nrf2 activator being a compound represented by Formula I or a pharmaceutically acceptable salt thereof: 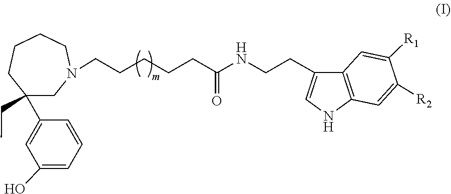
wherein
m is independently selected from an integer from 0 to 4, and
R₁ and R₂ are independently selected from hydrogen, hydroxyl, halogen, C₁₋₃ alkyl, C₁₋₃ alkoxy, halogen-substituted C₁₋₃ alkyl, or halogen-substituted C₁₋₃ alkoxy.

2. The Nrf2 activator according to claim 1, wherein the Nrf2 activator is used for preparing a medicament for treatment and/or prevention of diseases mediated by a Keap1-Nrf2/ARE signaling pathway and/or diseases mediated by oxidative stress.

3. The Nrf2 activator according to claim 2, wherein the Nrf2 activator is used for preparing a medicament for the treatment and/or prevention of amyotrophic lateral sclerosis, Friedrich's ataxia, multiple sclerosis, apoplexia, follicular abnormal development, premature ovarian failure, or infertility.

4. The Nrf2 activator according to claim 1, wherein m is 2 or 3, and R₁ is selected from hydroxyl, halogen, or C₁₋₃ alkoxy.

5. A method for treating and/or preventing diseases mediated by a Keap1-Nrf2/ARE signaling pathway and/or diseases mediated by oxidative stress using an Nrf2 activator, wherein
the Nrf2 activator is a compound represented by Formula I or a pharmaceutically acceptable salt thereof: 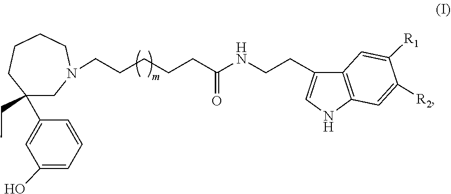
m is independently selected from an integer from 0 to 4, and
R₁ and R₂ are independently selected from hydrogen, hydroxyl, halogen, C₁₋₃ alkyl, C₁₋₃ alkoxy, halogen-substituted C₁₋₃ alkyl, or halogen-substituted C₁₋₃ alkoxy.

6. The method according to claim 5, wherein the diseases are amyotrophic lateral sclerosis, Friedrich's ataxia, multiple sclerosis, apoplexia, follicular abnormal development, premature ovarian failure, or infertility.
